(19) United States
(12) Patent Application Publication
(10) Pub. No.: US 2013/0102683 A1
(43) Pub. Date: Apr. 25, 2013

(54) MELT-GRANULATED FINGOLIMOD

(71) Applicants: Jana Paetz, Bonn (DE); Sandra Brueck, Munchen (DE)

(72) Inventors: Jana Paetz, Bonn (DE); Sandra Brueck, Munchen (DE)

(73) Assignee: RATIOPHARM GMBH, Ulm (DE)

(21) Appl. No.: 13/642,170

(22) PCT Filed: Apr. 21, 2011
(86) PCT No.: PCT/EP2011/002053

§ 371 Date: Dec. 23, 2012

(30) Foreign Application Priority Data
Apr. 22, 2010 (DE) 10 004 283.7

(51) Int. Cl. A61K 31/137; A61J 3/10; A61J 3/00

(52) U.S. Cl. CPC A61K 31/137; A61J 3/00; A61J 3/10
USPC 514/653; 427/2.14

(57) ABSTRACT

The invention relates to methods including the step of joint melt processing of (i) fingolimod or a pharmaceutically acceptable salt thereof, with (ii) a matrix former into an intermediate, intermediates obtainable in this way, and oral dosage forms, especially tablets, containing the intermediates of the invention. The invention further relates to a method of preparing the dosage forms of the invention, especially tablets. Finally, the invention relates to oral dosage forms for the treatment of multiple sclerosis.

The invention relates to a method involving the step of jointly melt-processing (i) fingolimod or a pharmaceutically acceptable salt thereof with (ii) a matrix former into an intermediate, intermediates obtainable in this way, and oral dosage forms, especially tablets, containing the intermediates of the invention. The invention further relates to a method of preparing the dosage forms of the invention, especially tablets. Finally, the invention relates to oral dosage forms for the treatment of multiple sclerosis.

Fingolimod, which is also known as “FTY720”, is a synthetic imitation of myriocin, a metabolic product from the fungus Isaria sinclairii. Fingolimod is a modulator of the sphingosine-1-phosphate receptor, which can bind, after phosphorylation, to sphingosine-1-phosphate receptors, especially T and B-lymphocytes. This inhibits the migration of lymphocytes from the lymph nodes into the blood and hence reduces their distribution in the central nervous system. Inflammatory T-lymphocytes are possible triggers for the destruction of the neural myelin sheaths, which are responsible for the typical symptoms of multiple sclerosis. For this reason, fingolimod is a possible means for the treatment of multiple sclerosis and especially for the treatment of patients with relapsing-remitting multiple sclerosis.

The IUPAC name of fingolimod is 2-amino-2-(2-[4-octylphenyl]ethyl)-1,3-propane diol. The chemical structure of fingolimod is shown in formula (1) below:

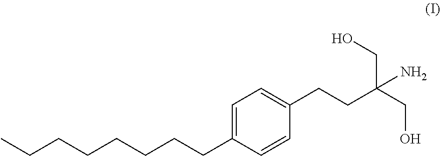 [SMILES: CCCCCCCCc1ccc(CCC(N)(CO)CO)cc1]

The synthesis of fingolimod is described in, for example, the European patent application EP 0 627 406.

Fingolimod is currently undergoing Phase III clinical trials, in which doses of 0.5 and 1.25 mg are being administered orally once a day. For the treatment of multiple sclerosis, doses ranging from 0.25 to 2.5 mg, i.e. very small amounts, are generally contemplated.

The proportion of the active agent in the total weight of the formulation (incl. active agent), or the formulation unit, especially in the case of formulations for oral administration, is typically in the range of only a few percent by weight, such as 0.25 to 4% by weight. This small proportion of active agent can lead to considerable problems during the manufacture of the formulation with regard to the uniformity of the content of active agent in the individual formulation units. For example, minor changes in the content of active agent, perhaps caused by changes in the flowability, especially of the active agent, and/or separation phenomena can lead to major variations.

The Ph. Eur. 6.0 section 2.9.6 therefore prescribes a uniformity test for the content of active agent in formulation units. According to that test, each individual content of 10 units must lie between 85 and 115 percent of the average content. If more than one individual content lies outside that limit or if one individual content lies outside the limits of 75 to 125 percent of the average content, the formulation units do not pass the test.

In addition, experiments on various salts of fingolimod have shown that depending on the ambient conditions, the possibility of water adsorption exists. Experiments with, for example, the hydrochloride of fingolimod showed that when the atmospheric humidity was set at 75% by means of a hygrostat, the water content increased 7-fold after 2 weeks. Water adsorption on this scale is detrimental to the storage stability of dosage forms containing fingolimod, especially in the case of tablets, granules or powders.

One problem to be solved by the present invention therefore consists in providing an oral dosage form containing fingolimod which exhibits good uniformity (homogeneity) of the content of active agent, and also a method of preparing it.

A further problem of the present invention consists in providing an oral dosage form of fingolimod which exhibits good storage stability with regard to the uniformity of the content of active agent.

One problem of the present invention consists especially in providing an oral dosage form containing fingolimod whose content of active agent, especially also after an extended storage time, lies within the concentration limits of 85 and 115 percent and preferably 90 and 110 percent of the average content according to Ph. Eur.

A further problem of the present invention consists in providing a method which makes it possible to prepare a dosage form containing fingolimod or fingolimod salt, avoiding the use of solvents, especially water.

It has surprisingly been possible to solve these problems by means of an intermediate obtainable by melt processing, an oral dosage form containing that intermediate and methods of preparing it.

One subject matter of the present invention is a method of preparing an intermediate containing fingolimod, comprising melt processing

(i) fingolimod or a pharmaceutically acceptable salt thereof, with
(ii) a matrix former.

A further subject matter of the present invention is accordingly an intermediate which is obtainable by melt processing (i) fingolimod or a pharmaceutically acceptable salt thereof, with (ii) a matrix former.

Further pharmaceutical excipients may optionally be used in the melt processing, as are described below. The intermediate may accordingly contain one or more pharmaceutical excipients in addition to the matrix former. Embodiments are, however, also encompassed in which only fingolimod and matrix former are contained in the intermediate.

An oral dosage form is a further subject matter of the present invention. It is preferably in the form of a tablet and contains:

(α) the intermediate of the invention and
(β) pharmaceutical excipients.

The oral dosage form can be designed for immediate release (or “IR” for short) or modified release (or “MR” for short).

A further subject matter of the present invention is a method of preparing the oral dosage form of the invention in the form of a tablet, comprising the steps of

(a) melt processing (i) fingolimod or one of its pharmaceutically acceptable salts, with (ii) a matrix former and optionally further pharmaceutical excipients into an intermediate;
(b) optionally granulating the intermediate;
(c) compressing the resulting intermediate into tablets, optionally with the addition of further pharmaceutical excipients; and
(d) optionally film-coating the tablets.

A further subject matter of the present invention is an oral dosage form of the invention, e.g. for immediate release, containing fingolimod for the treatment of multiple sclerosis, preferably relapsing-remitting multiple sclerosis.

In the context of the present invention, the term “fingolimod” comprises 2-amino-2-(2-[4-octylphenyl]ethyl)-1,3-propane diol according to the above formula (I). In addition, the term “fingolimod” comprises all the pharmaceutically acceptable salts, hydrates and/or solvates thereof. The salts used are preferably acid addition salts. Examples of suitable salts are hydrochlorides, carbonates, hydrogen carbonates, acetates, lactates, butyrates, propionates, sulphates, methane sulphonates, citrates, tartrates, nitrates, sulphonates, oxalates and/or succinates. Fingolimod hydrochloride is particularly preferably used.

Fingolimod is preferably present in the intermediate of the invention in particulate, preferably crystalline, form. In other words, there are preferably particles or crystals of fingolimod present with a size of preferably at least 100 nm, preferably at least 200 nm, or at least 300 nm embedded in a matrix which contains the matrix former or consists of the matrix former.

This is preferably fingolimod in crystalline form. In other words, preferably more than 90% by weight of fingolimod is present in crystalline form, particularly preferably 100% by weight of fingolimod is present in crystalline form.

The crystalline character or crystalline proportion of fingolimod can be determined with the aid of quantitative x-ray diffractometry using the evaluation method according to Hermans and Weidinger.

It has transpired that thanks to their good flowability, bulk density and compressibility, the intermediates of the invention are very advantageous in their use for preparing pharmaceutical formulations.

In addition, it has surprisingly been found that by using pharmaceutical formulations containing the intermediates of the invention, dependencies of the absorption of the active agent on the intake of food (“food effect”) can be eliminated or at least reduced substantially. The intermediates of the invention and the pharmaceutical formulations containing them can release the active agent independently of the pH.

Another particular advantage of these intermediates and the dosage forms containing them is that they can advantageously be administered with other medicaments, i.e. pharmaceutical formulations with an active agent different from fingolimod, without the absorption of fingolimod being impaired. This applies especially to medicaments which are suitable for influencing the pH at the site where active agent is absorbed.

In the context of this invention, the “matrix former” (ii) is generally a substance or mixture of substances which, when heated above the melting point, especially in a melt granulation or melt extrusion process, is deformable and capable of embedding particulate fingolimod, i.e. of forming a matrix for particulate fingolimod. Hence, the matrix former preferably exhibits thermoplastic behaviour, i.e. it is a thermoplastic matrix former. Furthermore, in this context, the matrix former is preferably a substance or mixture of substances which is capable of being deposited (chemically or physically) during the extrusion process on fingolimod or salts thereof and of increasing the hydrophilicity of the surface.

The matrix former (ii) may be a hydrophilic polymer, especially a hydrophilic thermoplastic polymer or mixtures thereof. Hydrophilic polymers are polymers which possess hydrophilic groups. Examples of suitable hydrophilic groups are hydroxy, alkoxy, amino, carboxy, sulphonate. In addition the hydrophilic polymer which can be used for the preparation of the intermediate preferably has a weight-average molecular weight of 1,000 to 150,000 g/mol, more preferably from 2,000 to 90,000 g/mol, especially 3,000 to 75,000 g/mol. The weight-average molecular weight is preferably determined in the context of this application by means of gel permeation chromatography.

When the polymer used as the matrix former is dissolved in water in an amount of 2% by weight, the resulting solution preferably has a viscosity of 0.1 to 8 mPa×s, more preferably 0.5 to 7 mPa×s, especially 1 to 6 mPa×s, measured at 25° C. and determined in accordance with Ph. Eur., 6th edition, chapter 2.2.10.

In addition to this, a hydrophilic polymer used as a matrix former preferably has a glass transition temperature (Tg) or a melt temperature (Ts) of at least 20° C., preferably higher than 20° C. to 220° C., more preferably 40° C. to 180° C., even more preferably 40° C. to 100° C. The glass transition temperature is the temperature at which the hydrophilic polymer becomes brittle when cooling down and soft when being heated. This means that hydrophilic polymers become soft at temperatures higher than the glass transition temperature (Tg) and become plastically deformable without breaking. The glass transition temperature or the melt temperature is determined by means of a Mettler-Toledo®DSC1, applying a heating rate of 10° C. per minute and a cooling rate of 15° C. per minute. The determination method is based essentially on Ph. Eur. 6.1, Chapter 2.2.34. In order to determine the Tg or the Ts, the polymer is heated twice (i.e. heated, cooled, heated).

In addition, the matrix former (ii) also includes solid, non-polymeric compounds which preferably contain polar side groups.

The intermediate of the invention may, for example, comprise the following hydrophilic polymers as matrix formers: polysaccharides, such as hydroxypropyl methyl cellulose (HPMC), polyvinyl pyrrolidone, polyvinyl alcohol, polymers of acrylic acid and their salts, polyacrylamide, polymethacrylates, vinyl pyrrolidone-vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), polyethylene oxide and mixtures of the polymers mentioned.

It is preferable to use as the matrix former (ii): hydroxypropyl methyl cellulose (HPMC), preferably with a weight-average molecular weight of 20,000 to 90,000 g/mol and/or preferably a proportion of methyl groups of 10 to 35%; hydroxypropyl cellulose (HPC), preferably with a weight-average molecular weight of 40,000 to 100,000 g/mol, polyvinyl pyrrolidone, preferably with a weight-average molecular weight of 10,000 to 60,000 g/mol, especially 12,000 to 40,000 g/mol, copolymer of vinyl pyrrolidone and vinyl acetate, especially with a weight-average molecular weight of 40,000 to 75,000 g/mol, polyethylene glycol, especially with a weight-average molecular weight of 2,000 to 50,000 g/mol, polyoxyethylene alkyl ether and/or polyvinyl alcohol, preferably with a weight-average molecular weight of 1,000 to 50,000 g/mol.

It is particularly preferable to use as the matrix former (ii) co-block polymers of polyethylene glycol and polypropylene glycol, i.e. polyoxyethylene polyoxypropylene block polymers. These preferably have a weight-average molecular weight of 1,000 to 20,000 g/mol, more preferably 1,500 to 12,500 g/mol, especially 5,000 to 10,000 g/mol. These block polymers are preferably obtainable by condensation of propylene oxide with propylene glycol and subsequent condensation of the polymer formed with ethylene oxide. This means that the ethylene oxide content is preferably present as an “endblock”. The block polymers preferably have a weight ratio of propylene oxide to ethylene oxide of 50:50 to 95:5, more preferably 70:30 to 90:10. The block polymers preferably have a viscosity at 25° C. of 200 to 2,000 mPa×s, more preferably 500 to 1,500 mPa×s, especially 800 to 1,200 mPa×s.

In the context of this invention, it is also possible to use mixtures of the above-mentioned examples of matrix formers. In one possible embodiment, a mixture of, for example, polyvinyl pyrrolidone and polyoxyethylene/polyoxypropylene block polymer is used.

As explained above, the matrix former preferably comprises or consists of a polymer or mixture of polymers. The matrix former may, however, also include substances which behave like polymers. Furthermore, the matrix former may also include solid, non-polymeric compounds which preferably contain polar side groups.

In a preferred embodiment, the intermediate of the invention contains fingolimod (or a pharmaceutically acceptable salt thereof), and matrix former, the weight ratio of active agent (i) fingolimod to matrix former (ii) in the context of the first embodiment being 5:1 to 1:150, more preferably 3:100 to 1:50, even more preferably 2:10 to 1:5.

It is preferable in the context of one embodiment that the type and amount of the matrix former are selected such that at least 50% of the surface of the resulting intermediate particles is covered with matrix former, more preferably at least 60% of the surface, particularly preferably at least 80% of the surface, especially at least 95% of the surface.

In the context of this invention, fingolimod (i) can be used as the sole active agent. Embodiments with one or more further active agents are, however, also encompassed by the present invention.

In a preferred embodiment, the fingolimod per se or a pharmaceutically acceptable salt thereof used in the dosage form has a water content of 0.01 to 10% by weight, more preferably 0.25 to 8.0% by weight, e.g. 0.27 to 7.5% by weight and particularly preferably 0.29 to 5% by weight. In the context of this application, the water content is preferably determined according to the Karl Fischer method, using a coulometer at 160° C. A Metrohm 831 KF coulometer with a titration cell without a diaphragm is preferably used. Usually, a 20 mg sample of fingolimod is analysed.

According to the present invention, an “intermediate” is preferably understood to mean a pharmaceutical composition which is not administered directly, but is instead converted into an applicable oral dosage form by means of suitable methods, such as granulation and/or compression.

In the context of this invention, fingolimod (i) and matrix former (ii) are “meltprocessed” jointly. It is preferable in this context that the melt processing is performed as melt extrusion or more preferably as melt granulation. During melt processing, it is also possible for further pharmaceutical excipients, such as disintegrants and wicking agents, to be added as described below for the oral dosage form. If disintegrants and wicking agents are contained within the intermediate, i.e. intragranularly, in the intermediate of the invention, they will be referred to in the context of this application as components (iii-int) or (iv-int). If disintegrants and wicking agents are contained outside the intermediate, i.e. extragranularly, in the oral dosage form of the invention, they will be referred to in the context of this application as components (iii-ex) or (iv-ex). If reference should be made to the total amount of disintegrants or wicking agents (i.e. both extragranular and intragranular), the designation (iii) or (iv) will be used.

The melt processing can be performed, as described below, in conventional melt processing apparatuses.

When crystalline fingolimod is used, the melting conditions can advantageously be selected such that fingolimod remains in a crystalline state.

The intermediate of the invention is used in the preparation of an oral dosage form. The oral dosage form is, for example, capsules, powder or granules for filling in sachets or tablets. The preparation of tablets is preferred in this context. It is particularly preferable for the intermediate of the invention to be used for preparing an immediate-release tablet.

As already mentioned, the subject matter of the invention is also an oral dosage form, especially a tablet, e.g. with immediate release, which contains
(α) intermediate of the invention and
(β) pharmaceutical excipients.

These are the excipients (β), with which the person skilled in the art is familiar, especially those which are described in the European Pharmacopoeia.

Examples of excipients (β) used are disintegrants, anti-stick agents, additives to improve the powder flowability, glidants, wetting agents and/or lubricants.

The ratio of active agent to excipients is preferably selected such that the resulting formulations contain 0.1 to 4% by weight, more preferably between 0.1 and 2.5% by weight, more preferably between 0.15 and 1.5% by weight, particularly preferably between 0.2 and 1.2% by weight, fingolimod, and 1 to 99.9% by weight, more preferably 55 to 99.85% by weight, pharmaceutically acceptable excipients. The fingolimod is preferably crystalline, as already described for the intermediate.

In these ratios specified, the amount of matrix former used to prepare the intermediate of the invention is counted as an excipient. This means that the amount of active agent refers to the amount of fingolimod contained in the finished oral dosage form.

In a preferred embodiment, the tablet of the invention, which is preferably designed for immediate release, contains 1 to 40% by weight, 5 to 35% by weight, more preferably 10 to 30% by weight, particularly preferably 15 to 25% by weight disintegrants (iii), based on the total weight of the formulation. “Disintegrants” is the term generally used for substances which accelerate the disintegration of a dosage form, especially a tablet, after it is placed in water. Suitable disintegrants are, for example, organic disintegrants such as carrageenan, celluloses and cellulose derivatives: croscarmellose, starches and starch derivatives: sodium carboxymethyl starch, polysaccharides: soya polysaccharides, alginates and crospovidone. In addition, inorganic disintegrants such as bentonites can be used. Alkaline disintegrants can likewise be used. The term “alkaline disintegrants” means disintegrants which, when dissolved in water, produce a pH level of more than 7.0. It is also possible to use mixtures of the above-mentioned disintegrants.

Crospovidone and/or croscarmellose are particularly preferably used as disintegrants, especially in the above-mentioned amounts.

In a preferred embodiment, the oral dosage form of the invention, preferably the tablet, contains 0 to 65% by weight, such as 1 to 60% by weight, more preferably 2 to 58% by weight, particularly preferably 5 to 55% by weight wicking agents (iv), based on the total weight of the formulation.

In general a wicking agent (iv) is a substance with the ability to draw up a biological fluid (preferably water) into a solid (preferably in the intermediates (i), preferably by means of physisorption). Physisorption is defined as a form of adsorption in which the fluid molecules can adhere to the surface of the wicking agent, preferably by means of van der Waals binding between the surface of the wicking agent and the adsorbed fluid molecule (preferably water). Normally a wicking agent achieves this with or without swelling. Normally, a non-swelling wicking agent which attracts water will ultimately have a volume consisting substantially of the volume of the wicking agent and the amount of water which it attracts. In general, a swelling wicking agent will have a volume consisting substantially of the volume of the wicking agent, the amount of water which it attracts, and an additional volume, caused by steric and molecular forces.

In the intermediate of the invention or in the oral dosage form of the invention, preferably a tablet, the wicking agent (iv) preferably causes the formation of channels or pores. This facilitates the penetration of the water molecules into the intermediates, especially by physisorption. The function of the wicking agent therefore consists in transporting water to the surfaces inside the intermediates in order in this way to create channels in or a network on an enlarged surface.

Examples of wicking agents used are: microcrystalline cellulose, silicified micro-crystalline cellulose, colloidal silica, kaolin, titanium dioxide, fumed silica, aluminium, niacinamide, M-Pyrol, bentonite, magnesium-aluminium silicate, polyester, polyethylene, or mixtures thereof. The wicking agents of the pharmaceutical composition of the present invention preferably contain magnesium aluminium silicates, preferably Al₂O₃MgO.1,7SiO₂xH₂O, e.g. Neusilin®, cellulose and cellulose derivatives, such as silicified micro-crystalline cellulose, colloidal silica, and mixtures thereof. The silicified micro-crystalline cellulose preferably used is commercially obtainable under the trade name Prosolv® and has a silica content of 1 to 3% by weight, preferably 2% by weight.

The distribution of disintegrants and wicking agents, if present, among the interior of the intermediate (intragranularly) and the surroundings of the intermediate (extragranularly) is dependent on the matrix former used. In a preferred embodiment, at least 10% by weight of the total amount of the excipients used in the oral dosage form, preferably tablet, are located in the interior of the intermediate, i.e. intragranularly.

The oral dosage form of the invention, especially a tablet, may also contain fillers (v). “Fillers” generally means substances which serve to form the body of the tablet in the case of tablets with small amounts of active agent (e.g. less than 60% by weight). This means that fillers “dilute” the active agents in order to produce an adequate tablet-compression mixture. The normal purpose of fillers, therefore, is to obtain a suitable tablet size.

Examples of preferred fillers are lactose, lactose derivatives, starch, starch derivatives, treated starch, chitin, cellulose and derivatives thereof, calcium phosphate, sucrose, calcium carbonate, magnesium carbonate, magnesium oxide, maltodextrin, calcium sulphate, dextrates, dextrin and/or dextrose, hydrogenated vegetable oil.

Other fillers that can be used are sugar alcohols and/or disaccharides, such as mannitol, sorbitol, xylitol, isomalt, glucose, fructose, maltose and mixtures thereof. The term “sugar alcohols” in this context also includes monosaccharides.

Fillers may be used in an amount of 0 to 99.9% by weight, such as at least 0.75 or 1% by weight, more preferably 5 to 90% by weight, especially 10 to 80% by weight, based on the total weight of the formulation.

The oral dosage form of the invention, preferably a tablet, may also contain additives to improve the powder flowability. One example of an additive to improve the powder flowability is disperse silicon dioxide, e.g. known under the trade name Aerosil®. Preferably, silicon dioxide is used with a specific surface area of 50 to 400 m²/g, determined by gas adsorption in accordance with Ph. Eur., 6th edition 2.9.26.

Additives to improve the powder flowability are generally used in an amount of 0.05 to 5% by weight, e.g. 0.1 to 4% by weight, based on the total weight of the formulation.

Lubricants can be used in addition. Lubricants are generally used in order to reduce sliding friction. In particular the intention is to reduce the sliding friction found during tablet pressing between the punch moving up and down in the die and the die wall, on the one hand, and between the edge of the tablet and the die wall, on the other hand. Suitable lubricants are, for example, stearic acid, adipic acid, sodium stearyl fumarate (Pruv®) and/or magnesium stearate.

Lubricants are generally used in an amount of 0.1 to 5% by weight, preferably 1.0 to 4% by weight, based on the total weight of the formulation.

Anti-stick agents can be used in addition. “Anti-stick agents” is usually understood to mean substances which reduce agglomeration in the core bed. Examples are talcum, silica gel, polyethylene glycol (preferably with 2,000 to 10,000 g/mol weight-average molecular weight) and/or glycerol monostearate.

It lies in the nature of pharmaceutical excipients that they sometimes perform more than one function in a pharmaceutical formulation. In the context of this invention, in order to provide an unambiguous delimitation, the fiction will therefore preferably apply that each substance performs only one function. I.e. a substance which is used as a particular excipient is not simultaneously also used as a further pharmaceutical excipient. Sorbitol, for example—if used as a filler—is not also counted as a matrix former in addition. Similarly, micro-crystalline cellulose—if used as a wicking agent—is not also used as a filler, for example (even though microcrystalline cellulose also exhibits a filling effect). To put it another way, two excipients with different functions, e.g. wicking agents and lubricants, should be different from one another in material terms, i.e. they should be formed from different substances.

In a preferred embodiment, the oral dosage form of the invention, preferably a tablet, comprises the following components (based on the total weight of the oral dosage form or tablet, without a film coating or the like):
0.1 to 4% by weight fingolimod (i),
0.75 to 99.9% by weight matrix former (ii), and
at least one pharmaceutical excipient (β) selected from the group comprising:
0.75 to 99.9% by weight, preferably 5 to 99% by weight filler (v),
1 to 35% by weight disintegrant (iii),
1 to 65% by weight wicking agent (iv),
0.1 to 5% by weight lubricant.

In a further preferred further embodiment, the oral dosage form of the invention, preferably a tablet, comprises the following components (based on the total weight of the oral dosage form or tablet, without a film coating or the like):
0.15 to 2.0% by weight fingolimod (i),
1.0 to 90% by weight matrix former (ii), and
at least one pharmaceutical excipient (β) selected from the group comprising:
1 to 90% by weight filler (v),
5 to 30% by weight disintegrant (iii)
1 to 60% by weight wicking agent (iv),
0.5 to 4.5% by weight lubricant.

In a further preferred further embodiment, the oral dosage form of the invention, preferably a tablet, comprises the following components (based on the total weight of the oral dosage form or tablet, without a film coating or the like):
0.2 to 1.5% by weight fingolimod (i),
1.0 to 80% by weight matrix former (ii), and
at least one pharmaceutical excipient (1) selected from the group comprising:
5 to 80% by weight filler (v),
10 to 25% by weight disintegrant (iii),
5 to 55% by weight wicking agent (iv) and
1.0 to 4.0% by weight lubricant.

A subject matter of the invention is, as already mentioned, a method of preparing the tablet of the invention, comprising the steps of:
(a) melt processing, preferably melt extruding or especially melt granulating,
   (i) fingolimod or its pharmaceutically acceptable salts, with (ii) a matrix former and optionally further pharmaceutical excipients into an intermediate;
(b) optionally granulating the intermediate;
(c) compressing the resulting intermediates (preferably the granules resulting from step (b)) into tablets, optionally with the addition of further pharmaceutical excipients; and
(d) optionally film-coating the tablets.

In principle, all the explanations given above on preferred embodiments of the intermediate of the invention also apply to the method of the invention, and vice versa.

In a preferred embodiment, before step (a) of the method of the invention or as part of step (a), (i) fingolimod is prepared and mixed with (ii) a matrix former and optionally further pharmaceutical excipients (1)—as described above.

In embodiments for immediate release, the matrix former preferably does not include any polymer with a weight-average molecular weight of more than 150,000 g/mol. The same applies to the pharmaceutical excipients added in step (a) (and/or also in step (d)) of the method of the invention.

The mixing can be performed in conventional mixers. For example, a Turbula® T10B (Bachofen AG, Switzerland) is suitable. The mixing time is usually 1 minute to 1 hour, preferably 5 minutes to 20 hours.

In a preferred embodiment, before step (a) (or as part of step (a)),
100% of the fingolimod used,
100% of the matrix former used,
optionally 20 to 70% of the filler used,
optionally 20 to 70% of the wicking agent used, and
optionally 30 to 70% of the disintegrant used, and
optionally 10 to 40% of the lubricant used are mixed. The remaining optional amounts of filler, disintegrant and lubricant are optionally added subsequently in step (c).

In step (a) of the method of the invention, a mixture of (i) fingolimod is meltprocessed, i.e. preferably melt-extruded or melt-granulated with (ii) a matrix former and optionally further pharmaceutical excipients (β), into the intermediate of the invention.

In one embodiment of the present invention, in the course of the melt processing, (a) fingolimod (i) is processed with the—preferably thermoplastic—matrix former (ii) in such a way that fingolimod is embedded in the matrix former. In this connection, it is preferable for the melting conditions to be selected such that the matrix former is melted or partially melted, whereas the active agent remains in a solid state. Fingolimod is preferably used in this context in crystalline form (especially as fingolimod hydrochloride) and the melting conditions are preferably selected such that the active agent remains in crystalline form.

The temperature chosen during the melt processing is preferably from 10° C. below to 10° C. above the melting point of the matrix former, preferably with the proviso that the temperature chosen is at least 10° C. below the melting temperature of the fingolimod used.

The melt processing can preferably be performed as melt granulation or as melt extrusion.

In a preferred embodiment, melt granulation is performed. In this case, the melting process is preferably performed by means of an intensive mixer with a heatable jacket unit; a Diosna® P1-6, for example, can advantageously be used. In this context, it is usual for the mixture of components (i) and (ii) to be premixed in a dry state without heating the jacket and then heated up in a second step by switching on the heatable jacket, preferably with stirring. The heating is preferably continued until an increase in the power consumption is observed. After that, the mixture is granulated and cooled.

In a different preferred embodiment, melt extrusion is performed. This is a continuous method (independent of batches), where the pre-mixing and granulating are not performed sequentially, but rather in one production step. A preferred method of preparing the melt extrudate is melt extrusion by means of a twinscrew extruder (e.g. Leistritz® micro 18). It is an advantage here that setting a temperature gradient, depending on the matrix former chosen, allows the dwell time of the fingolimod at high temperatures to be reduced considerably. The temperature gradient is usually between 80-190° C. and is preferably selected such that after processing, the fingolimod is still present in crystalline form if this is desired in the context of the first embodiment.

In the optional step (b) of the method of the invention, the extruded material is granulated. The granulating may take place before, during or after cooling. The granulating preferably already takes place in the course of the melt processing. In this way, steps (a) and (b), for example, can also be regarded as a single processing step.

In a preferred embodiment, the granulation conditions (in step (a) or step (b)) are selected such that the resulting particles (granules) have a weight-average particle size (D50 value) of 75 to 600 μm, more preferably 120 to 500 μm, even more preferably 150 to 400 μm, especially 200 to 350 μm. The weight-average particle size is determined by means of screen analysis (using a Retsch® AS 2000, amplitude 1.5 sec., interval 10 min., amount of sample 15.8 g).

In addition, the granulation conditions are preferably selected such that the resulting granules have a bulk density of 0.3 to 0.85 g/ml, more preferably 0.4 to 0.8 g/ml, especially 0.4 to 0.7 g/ml. The Hausner factor is usually in the range from 1.02 to 1.3, more preferably from 1.03 to 1.25 and especially from 1.04 to 1.15. The “Hausner factor” in this context means the ratio of tapped density to bulk density. The bulk density and tapped density are determined in accordance with USP 24, test 616 “Bulk Density and Tapped Density”.

In step (c) of the method of the invention, the intermediates, or granules, obtained in steps (a) or (b) are pressed into tablets, i.e. the step involves compression into tablets. The compression can be performed with tableting machines known in the prior art, such as eccentric presses or rotary presses. In the case of rotary presses, a compressive force of 2 to 40 kN, preferably 2.5 to 35 kN, is usually applied. As an example, the Fette® 102i press (Fette GmbH, Germany) is used. In the case of eccentric presses, a compressive force of 1 to 20 kN, preferably 2.5 to 10 kN, is usually applied. By way of example, the Korsch® EK0 is used.

Process step (c) is preferably performed in the absence of solvents, especially organic solvents, i.e. as dry compression.

In step (c) of the method of the invention, pharmaceutical excipients (β) may be added to the intermediates, or granules, from steps (a) or (b). On this subject, reference may be made to the above explanations concerning suitable excipients

The subject matter of the invention is not only the method of the invention, but also the oral dosage forms, especially tablets, produced with this method.

The tablets produced by the method of the invention may be tablets which can be swallowed unchewed (non-film-coated or preferably film-coated). They may likewise be chewable tablets or dispersible tablets. “Dispersible tablet” here means a tablet to be used for producing an aqueous suspension for swallowing.

In the case of tablets which are swallowed unchewed, it is preferable that they be coated with a film layer in step (d) of the method of the invention. The above-mentioned ratios of active agent to excipient, however, relate to the non-film-coated, or uncoated, tablet.

For film-coating, macromolecular substances are preferably used, such as modified celluloses, polymethacrylates, polyvinyl pyrrolidone, polyvinyl acetate phthalate, zein and/or shellac.

HPMC is preferably used, especially HPMC with a weight-average molecular weight of 10,000 to 150,000 g/mol and/or an average degree of substitution of —OCH₃ groups of 1.2 to 2.0.

The thickness of the coating is preferably 1 to 100 μm, more preferably 2 to 80 μm.

The tableting conditions are preferably selected such that the resulting tablets have a tablet height to weight ratio of 0.003 to 0.03 mm/mg, more preferably 0.004 to 0.02 mm/mg, particularly preferably 0.004 to 0.015 mm/mg.

In addition, the resulting tablets preferably have a hardness of 40 to 200 N, particularly preferably 60 to 150 N, especially if the tablet weight is more than 200 mg. If the tablet weight is 200 mg or less, the resulting tablets preferably have a hardness of 30 to 130 N, particularly preferably 40 to 100 N. The hardness is determined in accordance with Ph. Eur. 6.0, section 2.9.8.

In addition, the resulting tablets preferably have a friability of less than 3%, particularly preferably less than 1%, especially less than 0.8%. The friability is determined in accordance with Ph. Eur. 6.0, section 2.9.7.

In addition, the intermediates and oral dosage forms of the invention, especially tablets, exhibit a high degree of uniformity of the content of active agent. In order to determine the uniformity of the intermediates, 20 individual samples with a volume of 10 ml each are taken from the intermediate at random. The uniformity of the content of active agent is then determined in accordance with Ph. Eur. 6.0, Chapter 2.9.6, HPLC being used as the analytical process. It is preferable for each of twenty individual samples of the intermediate to have a fingolimod content of between 90% and 110%, preferably 92% to 108%, even more preferably 94% to 106%, particularly preferably 96% to 104% and especially 98% to 102% of the average content of those twenty individual samples. It is accordingly preferable for each of twenty dosage forms (or units), especially tablets, to have a fingolimod content of between 90% and 110%, preferably 92% to 108%, even more preferably 94% to 106%, particularly preferably 96% to 104% and especially 98% to 102% of the average content of those twenty dosage forms. Intermediates and dosage forms with such uniformity are preferred embodiments of the present invention.

In the case of an IR formulation, the release profile of the tablets of the invention according to the USP method (USP basket apparatus, 500 ml test medium; 0.1 N HCl and 0.2% sodium dodecyl sulfate, 37° C. and 100 rpm) after 10 minutes usually indicates a content released of at least 30%, preferably at least 60%, especially at least 98%.

In the case of a MR formulation, the release profile of the tablets of the invention according to the USP method (USP basket apparatus, 500 ml test medium; 0.1 N HCl and 0.2% sodium dodecyl sulfate, 37° C. and 100 rpm) after 10 minutes indicates, for example, a content released of no more than 98%, preferably no more than 90%, further preferably no more than 75%, more preferably no more than 50% and particularly preferably no more than 30%.

The above details regarding hardness, friability, content uniformity and release profile preferably here relate to the non-film-coated tablet for an IR formulation. For a modified release tablet, the release profile relates to the total formulation.

As an alternative to compression into tablets, the granules resulting in step (c) of the method of the invention may also be processed—optionally with the addition of further pharmaceutical excipients—into a particulate dosage form, such as by filling into capsules or sachets.

A further subject matter of the present invention is an oral dosage form of the invention containing fingolimod for the treatment of multiple sclerosis, preferably relapsing-remitting multiple sclerosis.

A further advantage of the oral dosage form of the invention is that it can be administered independently of meal times. In a preferred embodiment for immediate release and administration independently of meal times, a disintegrant is used in an amount of 10 to 30% by weight, based on the total weight of the oral dosage form. In a further preferred embodiment, a polyoxyethylene polyoxypropylene block polymer is used as the matrix former for this purpose, especially as described in more detail above.

The invention will now be illustrated with reference to the following examples.

### EXAMPLES

### Example 1a

### Preparation of an Intermediate by Melt Granulation and Subsequent Compression into Tablets

0.5 g fingolimod and 2.5 g polyoxyethylene polyoxypropylene block polymer (ratio of fingolimod to polymer=1:5), 20.0 g micro-crystalline cellulose and 10.0 g crospovidone were granulated with gentle warming to the melting point of the polymer, and an intermediate was prepared in this way. The resulting intermediate was screened (mesh width 0.6 mm) and mixed thoroughly for a further 10 minutes.

The intermediate (granules) was then mixed for 10 minutes together with the remaining 18.0 g crospovidone and 50.0 g sorbitol, 80.0 g micro-crystalline cellulose and 0.2 g Aerosil. 7.0 g sodium stearyl fumarate was added through a screen (mesh width 0.3 mm) and the resulting mixture mixed for a further 5 minutes and then compressed into tablets.

The tablet produced in this way, or the tablet core produced in this way, had the following composition:

| | | |
|---|---|---|
| fingolimod | 0.5 | mg |
| polyoxyethylene polyoxypropylene | 2.5 | mg |
| block polymer (Mw approx. 8350) | | |
| sorbitol (filler) | 50.0 | mg |
| sodium stearyl fumarate | 7.00 | mg |
| crospovidone | 28.0 | mg |
| microcrystalline cellulose | 100 | mg |
| highly disperse silica | 0.2 | mg |

The tablet produced in this way was then coated with an Opadry® AMB solution and thus film-coated:

| | |
|---|---|
| Opadry ® AMB | 10.40 mg |

### Example 1b

Tablets were produced according to Example 1a, except that the excipients microcrystalline cellulose and sorbitol were substituted by Neusilin® and α-lactose monohydrate. Thus, the tablet produced in this way or the tablet core produced in this way had the following composition:

| | | |
|---|---|---|
| fingolimod | 0.5 | mg |
| polyoxyethylene polyoxypropylene | 2.5 | mg |
| block polymer (Mw approx. 8350) | | |
| α-lactose monohydrate (filler) | 50.0 | mg |
| sodium stearyl fumarate | 7.00 | mg |
| crospovidone | 28.0 | mg |
| Neusilin ® (wicking agent) | 100 | mg |
| highly disperse silica | 0.2 | mg |

The tablet produced in this way was then coated with an Opadry® AMB solution and thus film-coated:

| | |
|---|---|
| Opadry ® AMB | 10.4 mg |

### Example 2a

### Preparation of an Intermediate by Melt Granulation and Subsequent Compression into Tablets

0.5 g fingolimod and 2.5 g polyoxyethylene polyoxypropylene block polymer (ratio of fingolimod to polymer=1:5), 20.0 g micro-crystalline cellulose and 10.0 g crospovidone were granulated with gentle warming to the melting point of the polymer, and an intermediate was prepared in this way. The resulting intermediate was screened (mesh width 0.6 mm) and mixed thoroughly for a further 10 minutes.

The intermediate (granules) was then mixed for 10 minutes together with the remaining 18.0 g crospovidone and 50.0 g sucrose, 80.0 g micro-crystalline cellulose and 0.2 g Aerosil. 7.0 g sodium stearyl fumarate was added through a screen (mesh width 0.3 mm) and the resulting mixture mixed for a further 5 minutes and then compressed into tablets.

The tablet produced in this way, or the tablet core produced in this way, had the following composition:

| | | |
|---|---|---|
| fingolimod | 0.5 | mg |
| polyoxyethylene-polyoxypropylene | 2.5 | mg |
| block polymer (Mw approx. 8350) | | |
| sucrose (filler) | 50.0 | mg |
| sodium stearyl fumarate | 7.00 | mg |
| crospovidone | 28.0 | mg |
| microcrystalline cellulose | 100 | mg |
| highly disperse silica | 0.2 | mg |

The tablet produced in this way was then coated with an Opadry® AMB solution and thus film-coated:

| | |
|---|---|
| Opadry ® AMB | 10.40 mg |

### Example 2b

Tablets were produced according to Example 2a, except that the excipient microcrystalline cellulose was substituted by Neusilin®. Thus, the tablet produced in this way or the tablet core produced in this way had the following composition:

| | | |
|---|---|---|
| fingolimod | 0.5 | mg |
| polyoxyethylene-polyoxypropylene | 2.5 | mg |
| block polymer (Mw approx. 8350) | | |
| sucrose (filler) | 50.0 | mg |
| sodium stearyl fumarate | 7.00 | mg |
| crospovidone | 28.0 | mg |
| Neusilin ® (wicking agent) | 100 | mg |
| highly disperse silica | 0.2 | mg |

The tablet produced in this way was then coated with an Opadry® AMB solution and thus film-coated:

| | |
|---|---|
| Opadry ® AMB | 10.4 mg |

### Example 3

### Preparation of an Intermediate by Melt Granulation and Subsequent Filling into Capsules

0.5 g fingolimod and 4.5 g Pluronic® F68, a polyoxyethylene polyoxypropylene block polymer (ratio of fingolimod to polymer=1:9), 18.0 g crospovidone and 36.0 g MCC were granulated by gently warming to the melting point of the polymer and an intermediate was prepared in this way. The resulting intermediate was screened (mesh width 0.6 mm) and mixed thoroughly for a further 10 minutes.

The intermediate (granules) was filled into capsules, each capsule having the following composition:

| | | |
|---|---|---|
| fingolimod | 0.5 | mg |
| Pluronic ® F68 | 4.5 | mg |
| Crospovidone | 18.0 | mg |
| microcrystalline cellulose | 36.0 | mg |

1. A method of preparing an intermediate, comprising melt processing
(i) fingolimod or a pharmaceutically acceptable salt thereof, with
(ii) a matrix former.

2. An intermediate, obtainable in accordance with claim 1, wherein the fingolimod or the pharmaceutically acceptable salt thereof is present in particulate, crystalline, form.

3. The intermediate as claimed in claim 2, wherein hydrophilic polymers with a weight-average molecular weight of 1,000 g/mol to 150,000 g/mol are used as matrix formers.

4. The intermediate as claimed in claim 2, wherein polyoxyethylene-polyoxypropylene block polymers, with a weight-average molecular weight of 1,500 to 12,500 g/mol, are used as matrix formers.

5. The intermediate as claimed in claim 4 wherein the weight ratio of component (i) to component (ii) is 5:1 to 1:150.

6. The intermediate as claimed in claim 2, further comprising
(iii-int) disintegrants and/or
(iv-int) wicking agents.

7. An oral dosage form, preferably in the form of a tablet, comprising
(α) an intermediate in accordance with claim 2 and
(β) pharmaceutical excipients.

8. The oral dosage form as claimed in claim 7, characterised in that the component (β) comprises disintegrants (iii-ex) and/or an wicking agent (iv-ex).

9. The oral dosage form as claimed in claim 8, wherein the total amount of disintegrants (iii-int)+(iii-ex) is 10 to 30% by weight, based on the total weight the formulation.

10. The oral dosage form as claimed in claim 7, wherein the oral dosage form has a fingolimod content of 0.1 to 4% by weight.

11. A method of preparing an oral dosage form in the form of a tablet, comprising the steps
(a) melt processing (i) fingolimod or one of its pharmaceutically acceptable salts, with (ii) a matrix former and optionally further pharmaceutical excipients, into an intermediate;
(b) optionally granulating the intermediate;
(c) compressing the resulting intermediate into tablets, optionally with the addition of further pharmaceutical excipients; and
(d) optionally film-coating the tablets.

12. The method as claimed in claim 11, wherein the melting conditions in step (a) are selected such that fingolimod remains in a crystalline state.

13. The method as claimed in claim 11, wherein in steps (a) or (b), granules with a weight-average particle size of 120 to 500 μm are produced.

14. An oral dosage form containing fingolimod in accordance with claim 7 for the treatment of multiple sclerosis.
